(19) United States
(12) Patent Application Publication
(10) Pub. No.: US 2010/0297717 A1
(43) Pub. Date: Nov. 25, 2010

(54) METHOD OF PRODUCING ALCOHOL IN THE BIOREFINERY CONTEXT

(75) Inventors: Antoine Margeot, Paris (FR); Frederic Monot, Nanterre (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: IFP, Rueil-Malmaison Cedex (FR)

(21) Appl. No.: 12/743,410

(22) PCT Filed: Nov. 6, 2008
(86) PCT No.: PCT/FR2008/001566

§ 371 Date: Aug. 10, 2010

(30) Foreign Application Priority Data
Nov. 21, 2007 (FR) 07/08173

(51) Int. Cl. C12P 7/16; C12P 7/02

(52) U.S. Cl. 435/160; 435/155

(57) ABSTRACT

The present invention describes a method of producing alcohol from pretreated lignocellulosic biomass wherein the enzymatic hydrolysis stage is carried out with cellulolytic and/or hemicellulolytic enzymes produced using at least one effluent from another ethanol production process using a sugar plant as the feedstock. This method can be integrated in a method of producing alcohol from cellulosic or lignocellulosic materials, referred to as second-generation method, comprising the following stages:
1) chemical and/or physical pretreatment of a cellulosic or lignocellulosic substrate,
2) enzymatic hydrolysis of the pretreated substrate using cellulolytic and/or hemicellulolytic enzymes,
3) alcoholic fermentation by a suitable alcohol-producing microorganism of the hydrolysate from stage (2) until a fermentation must is obtained, and separation of the alcohol-producing microorganism used in stage (3), separation/purification of the alcohol.

### FIELD OF THE INVENTION

The present invention lies within the scope of biofuel production. It more particularly relates to the production of cellulolytic and/or hemicellulolytic enzymes within the scope of alcohol production from cellulosic or lignocellulosic materials (second-generation biofuel).

The current economic and logistic principles require that the second-generation biofuel production sites be the same as the first-generation production sites, thus forming a “biorefinery” where all of the vegetable feedstock is upgraded. Starting from a sugar plant, the goal therefore is to upgrade sugar cane and lignocellulosic cane residues.

### BACKGROUND OF THE INVENTION

Since the 1970s, the conversion of lignocellulosic biomass to ethanol, after hydrolysis of the constituent polysaccharides to fermentable sugars, has been the subject of considerable research work.

Most lignocellulosic residues consist of approximately 40 to 50% cellulose, 20 to 25% hemicellulose and 15 to 25% lignin.

Among these three polymers, cellulose, hemicellulose and lignin, cellulose is the main source of ethanol-fermentable sugars because it consists of glucose, which can be readily fermented to ethanol by Saccharomyces cerevisiae in proven and efficient industrial processes. The pentoses contained in the hemicelluloses are not efficiently converted to ethanol. Other microorganisms among the Saccharomyces, Pichia, Candida, Pachysolen, Zymomonas, Klebsiella, Escherichia genera can be selected for upgrading the monomer sugars from the biomass to ethanol.

The method of converting lignocellulosic materials to alcohol comprises a physico-chemical pretreatment stage, followed by an enzymatic or chemical hydrolysis stage, a stage of alcoholic fermentation of the sugars released and an alcohol recovery stage.

The goal of the pretreatment stage is to release the sugars contained in the hemicelluloses as monomers, essentially pentoses, such as xylose and arabinose, and hexoses, such as galactose, mannose and glucose, and to improve the accessibility of the cellulose stuck in the lignin and hemicellulose matrix. There are many technologies: acidic boiling, alkaline boiling, steam explosion, organosolv processes, etc. The pretreatment efficiency is measured by the hemicellulose recovery rate and by the hydrolysis susceptibility of the cellulosic residue. Acidic pretreatments under mild conditions and steam explosion are the best suited techniques. They allow total recovery of the pentoses and good accessibility of the cellulose to hydrolysis.

The cellulosic residue is hydrolyzed either via the acidic process or via the enzymatic process using cellulolytic and/or hemicellulolytic enzymes. Microorganisms such as fungi belonging to the Trichoderma, Aspergillus, Penicillium or Schizophyllum genera, or anaerobic bacteria belonging for example to the Clostridium genus, produce these enzymes containing notably cellulases and xylanases, suited for total hydrolysis of the polymers that make up the plants.

The acidic process, carried out with a strong acid, notably sulfuric acid, is efficient but it requires large amounts of chemical products (acid, then base for neutralization). Enzymatic hydrolysis does not entail this drawback; it is carried out under mild conditions and it is efficient. On the other hand, the cost of enzymes is still very high. Considerable work has therefore been conducted in order to reduce this cost: first increase in the production of enzymes by selecting hyperproductive strains and by improving fermentation methods, then decrease in the amount of enzymes in hydrolysis by optimizing the pretreatment stage or by improving the specific activity of these enzymes. During the last decade, the main work consisted in trying to understand the mechanisms of action of the cellulases and of expression of the enzymes so as to cause excretion of the enzymatic complex that is best suited for hydrolysis of the lignocellulosic substrates by modifying the strains with molecular biology tools.

The most commonly used microorganism for cellulase production is the Trichoderma reesei fungus. Wild strains have the ability to excrete, in the presence of an inductive substrate, cellulose for example, the enzymatic complex that is considered to be the best suited for cellulose hydrolysis. The enzymes of the enzymatic complex comprise three major types of activities: endoglucanases, exoglucanases and cellobiases. Other proteins having properties that are essential for the hydrolysis of lignocellulosic materials are also produced by Trichoderma reesei, xylanases for example. The presence of an inductive substrate is essential to the expression of cellulolytic and/or hemicellulolytic enzymes. The nature of the carbon-containing substrate has a strong influence on the composition of the enzymatic complex. It is the case of xylose which, associated with an inductive carbon-containing substrate such as cellulose or lactose, allows the activity of xylanases to be significantly improved.

Conventional genetic mutation techniques have allowed cellulase-hyperproductive Trichoderma reesei strains such as MCG77 (Gallo—U.S. Pat. No. 4,275,167), MCG 80 (Allen, A. L. and Andreotti, R. E., Biotechnol-Bioengi 1982, 12, 451-459 1982), RUT C30 (Montenecourt, B. S. and Eveleigh, D. E., Appl. Environ. Microbiol. 1977, 34, 777-782) and CL847 (Durand et al., 1984, Proc. Colloque SFM “Génétique des microorganismes industriels”. Paris. H. HESLOT Ed, pp 39-50) to be selected. The improvements have allowed to obtain hyperproductive strains that are less sensitive to catabolic repression on monomer sugars notably, glucose for example, in relation to wild strains.

Recombinant strains have been obtained from the Trichoderma reesei strains Qm9414, RutC30, CL847 by cloning heterologous genes, for example the invertase from Aspergillus niger allowing Trichoderma reesei to use saccharose as a source of carbon. These strains have kept their hyperproductivity and their aptitude for being cultivated in fermenters.

The method of producing cellulases by Trichoderma reesei has been the subject of major improvements with a view to extrapolation to the industrial scale.

In order to obtain good enzyme productivities, it is necessary to provide a carbon source that can be rapidly assimilated for the growth of Trichoderma reesei and an inductive substrate allowing expression of cellulases and secretion in the culture medium. Cellulose can play both roles; however, it is difficult to use on an industrial scale and it has been replaced by soluble carbon sources such as glucose, xylose or lactose, lactose also acting as an inductive substrate. Other soluble sugars such as cellobiose and sophorose have been described as inductors, but they are too expensive to be used on an industrial scale. It has however been observed that cellulase productions by Trichoderma reesei, with soluble substrates, are much lower than those obtained on cellulose with a batch process. This is due to the repressing effect of readily assimilatable sugars, in high concentrations. Continuous supply of soluble carbon-containing substrates has allowed to lift the catabolic repression by limiting the residual concentration in the cultures and by optimizing the proportion of sugar allowing a higher yield and a better enzymatic productivity to be obtained (see patent FR-B-2,555,603 filed by the applicant).

Lactose remains one of the most suitable and less expensive substrates in an industrial cellulolytic enzyme production process; it is however still costly and represents about one third of the cost price of enzymes. Despite all the progress made, the cost of enzymes remains a significant item (30 to 50%) in the conversion of cellulosic biomass to alcohol. Furthermore, if lactose is used as the carbon source for cellulase production, the process depends on an external carbon source. The use of carbon-containing substrates from the industry (such as hydrolyzed hemicellulose residues or ethanolic fermentation vinasses) is therefore a great advance if the inductive carbon source is readily available.

The use of hydrolyzed hemicellulose residues for the production of cellulases has been described in patent application FR-A-2,881,753 filed on behalf of the applicant. These residues are actually cellulase production inductors and they lead to efficient mixtures for hydrolysis of lignocellulosic materials. The use of such residues however involves a certain number of potential limitations:

their use requires pretreatment wherein the hemicelluloses are solubilized,

vegetable feedstock variation such as, for example, a simple variety change may lead to a change in the composition of the residue and therefore to a change in the enzyme composition,

the presence of inhibitors, notably according to the vegetable and to the pretreatment conditions, may pose problems for culture reproducibility,

the high xylose content of the residues (from straw for example) inducts hemicellulases that are not necessarily required for hydrolysis of a substrate that has already been freed of the major part of its hemicelluloses.

On the other hand, the use of lignocellulosic material hydrolysate fermentation vinasses has also The performances obtained with this carbon source are equivalent to or even higher than those obtained with conventional substrates, but it is possible that the new enzyme generations with improved hydrolysis properties and the yeast strains suited to the fermentation conditions will reduce the amount of inductive carbon source in a proportion incompatible with an enzyme production.

Using a carbon source of reproducible composition, free of inhibitors, hardly or not depending on the feedstock to be treated, would be a great step forward. By remaining available on site, this carbon source also allows to develop the concept of biorefinery.

### SUMMARY OF THE INVENTION

The present invention describes a method of producing alcohol from pretreated lignocellulosic biomass wherein the enzymatic hydrolysis stage is carried out with cellulolytic and/or hemicellulolytic enzymes produced using at least one effluent from another ethanol production process using a sugar plant as the feedstock.

### BRIEF DESCRIPTION OF THE FIGURES

FIG. 1 describes a process flow diagram for conversion and production of alcohol according to the present invention.

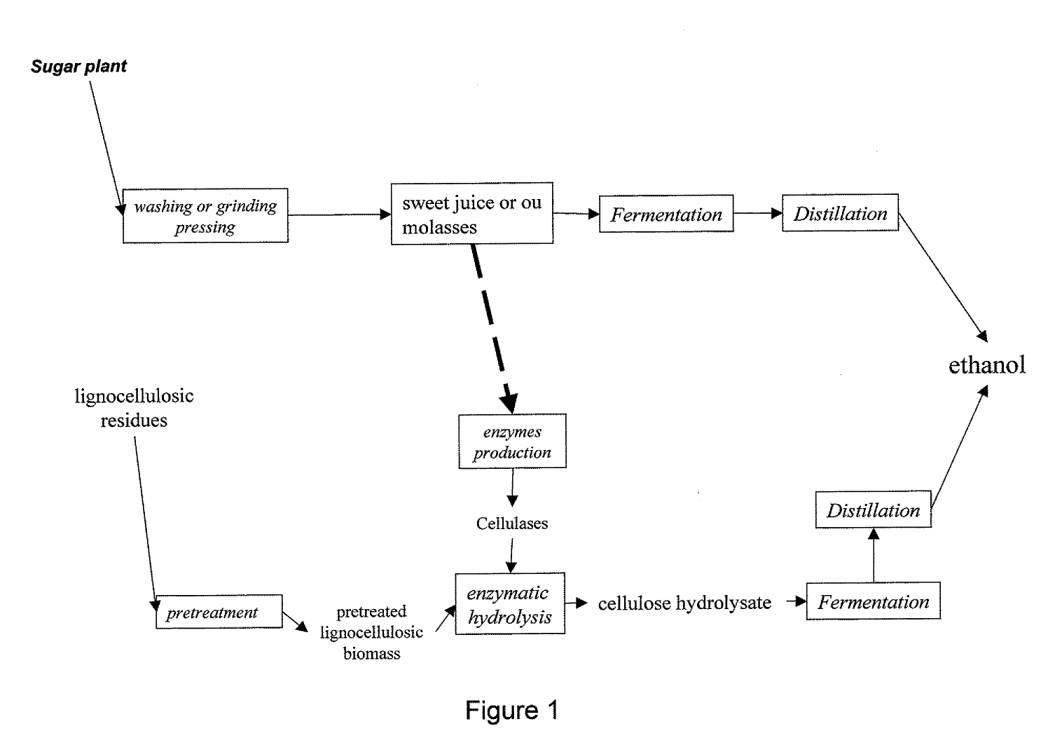

FIG. 2 describes a process flow diagram for conversion and production of alcohol according to the present invention using residues of a sugar plant as the lignocellulosic feedstock.

### DETAILED DESCRIPTION

The present invention relates to the use of effluents obtained during other biofuel production processes, by way of non limitative example the sweet juices resulting from beet or sugar cane washing, or sugar refinery molasses for the production of cellulolytic and/or hemicellulolytic enzymes, with cellulolytic microorganism strains using saccharose as the carbon source, naturally or after modification.

The cellulolytic microorganism belongs to the Trichoderma, Aspergillus, Penicillium or Schizophyllum genera, and preferably to the Trichoderma reesei species.

The sweet juices containing the saccharose used in the present invention come from first-generation biofuel production processes, notably from beet or sugar cane washing, or from sugar refinery molasses.

The main carbon source can be the saccharose obtained from these juices, or it can be a complement to an industrial soluble sugar, such as lactose or xylose, or a hemicellulosic fraction extract in form of monomers resulting from the pretreated biomass, or a fermentation vinasse extract from lignocellulosic biomass hydrolysis products, or a mixture thereof. The latter two extracts can also be used as cellulase production inductors.

One object of the invention is to provide a readily available carbon source allowing to produce cellulolytic and/or hemicellulolytic enzymes with activities suited for hydrolysis of cellulosic biomass, compatible with several bioalcohol production process schemes in various contexts.

The carbon source for the growth mainly of saccharose is obtained from sweet juices resulting from diffusion washing of beet pulps, or from molasses after sugar refining. Similarly, sweet juices or molasses resulting from cane sugar washing can be used.

The specific stage of preparation of the must from sacchariferous plants is extraction of the saccharose. This sugar is present in the free state in the vacuoles of the vegetable cells. It is extracted either by pressing or by countercurrent washing with hot water of the vegetable that has been cut into pieces beforehand.

Thus, the beets are generally washed to remove the earth, then cut into cossettes. A sweet juice is then extracted by diffusion with hot water. This first juice is usually intended for ethanolic fermentation but it can be used, concentrated or not in syrup form, in the present invention as carbon source for cellulase production. Molasses, which are sugar refinery by-products containing up to 50% by weight of saccharose, can also be used (Table 2).

In the case of sugar cane, the canes are crushed and pressed with addition of hot water to solubilize the sugars. The sweet juice, possibly concentrated, is then treated in the same way as in the beet option.

TABLE 1
Composition, for information only, of the sweet
juices obtained from hot water diffusion of beets
and from crushing and pressing of sugar canes.

| | Beet sweet juice | Sugar cane sweet juice |
|---|---|---|
| Dry matter (wt. %) | 15 | 20 |
| Total sugars (% DM) | 94 | >90 |
| incl. Saccharose | 94 | 85 |
| (% DM) | | |
| incl. Glucose (% DM) | 0 | 7.5 |
| incl. Fructose (% DM) | 0 | 7.5 |
| Others (% DM) | 6 | <10 |

The proportion of impurities is generally reduced by salt filtering and/or precipitation.

TABLE 2
Chemical composition, for information only, of beet and sugar
cane molasses (sources: INRA and Yang et al. (2007)).

| | Beet molasses | Sugar cane molasses |
|---|---|---|
| Dry matter (wt. %) | 73 | 73 |
| Mineral matter (% DM) | 13 | 14 |
| Total nitrogen-containing | 15 | 6 |
| matter (% DM) | | |
| Total sugars (% DM) | 64 | 64 |
| incl. Saccharose (% DM) | 64 | 43 |
| incl. Glucose (% DM) | 0 | 10.5 |
| incl. Fructose (% DM) | 0 | 10.5 |
| Calcium (g/kg DM) | 3.7 | 7.4 |
| Phosphorus (g/kg DM) | 0.3 | 0.7 |
| Potassium (g/kg DM) | 82 | 40 |

The lignocellulosic biomass used is selected from among straws, wood, forest crops, alcohol-producing, sugar crop and cereal crop residues, paper industry residues, cellulosic and lignocellulosic material transformation products.

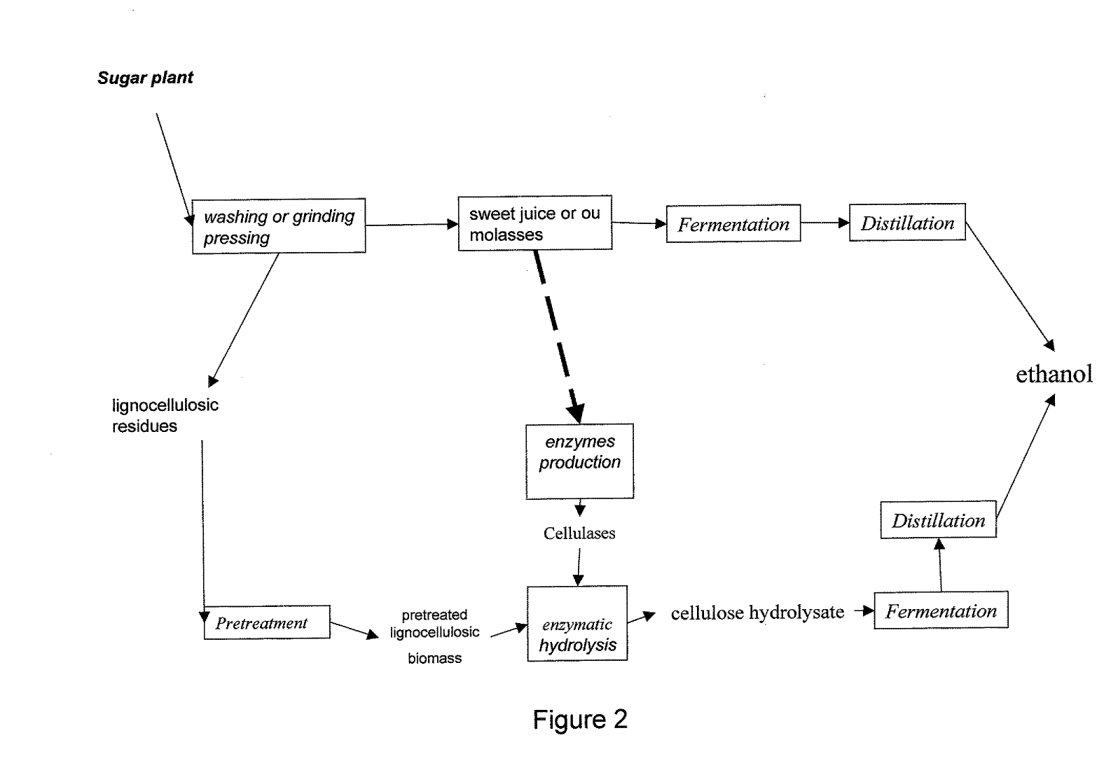

In the embodiment illustrated in FIG. 2, the lignocellulosic residues are derived from the sugar plant from which the sweet juices used for the production of cellulolytic and/or hemicellulolytic enzymes are obtained.

The lignocellulosic feedstock is subjected to a pretreatment that can be of any nature, with hydrolysis of the hemicelluloses or not. After pretreatment, the cellulosic fraction freed or not of the hydrolyzed hemicellulosic fraction, and possibly of the lignin, is hydrolyzed by the cellulolytic and/or hemicellulolytic enzymes produced by the specialized strains, the Trichoderma reesei cellulases being the most efficient and the most suitable when the carbon-containing substrates derive from cellulosic or lignocellulosic biomass. Said enzymes are produced using at least one effluent from another ethanol production process as described above.

The material to be hydrolyzed is suspended in the aqueous phase in a proportion of 6 to 40% dry matter, preferably 20 to 30%. The pH value is adjusted between 4 and 5.5, preferably between 4.8 and 5.2, and the temperature between 40° C. and 60° C., preferably between 45° C. and 50° C. The hydrolysis reaction is initiated by adding cellulases; the proportion commonly used ranges from 10 to 30 mg excreted proteins per gram of pretreated substrate or less. The reaction generally lasts for 15 to 48 hours depending on the pretreatment efficiency, the composition of the cellulase mixture and the amount of enzymes added. At the end of the reaction, the released sugars, notably glucose, are measured out. The sweet solution is separated from the non-hydrolyzed solid fraction essentially consisting of lignin, by filtering or centrifuging; it is used for ethanolic fermentation.

In general, the alcohol is separated from the fermentation must by distillation and the residue is made up of the distillation vinasses. These inductor-rich vinasses can be used as inductive carbon source for the production of cellulolytic and/or hemicellulolytic enzymes as described in patent application FR-A-2,881,753.

The strains used for the production of cellulolytic and/or hemicellulolytic enzymes are strains of fungi belonging to the Trichoderma, Aspergillus, Penicillium or Schizophyllum genera, preferably Trichoderma reesei. However, some of the fungi belonging to these genera, notably Trichoderma reesei, cannot use saccharose as the carbon source. It is therefore necessary to use genetically modified strains, for example expressing invertase from A. niger, as described for Trichoderma reesei by Berges et al., Curr. Genet. 1993 July-August 24 (1-2): 53-59, so that saccharose can be used as the carbon source. Independently, the strains can be modified to improve the cellulolytic and/or hemicellulolytic enzymes by means of mutation-selection processes such as, for example, the IFP CL847 strain (French patent FR-B-2,555,803); strains improved by genetic recombination techniques can also be used.

These strains are cultivated in stirred and aerated fermenters under conditions compatible with their growth and the production of enzymes. Depending on its nature, the carbon-containing substrate selected to obtain the biomass is fed into the fermenter prior to sterilization or it is sterilized separately and fed into the fermenter after sterilization thereof so as to have an initial concentration of 20 to 35 g.l⁻¹. The inductive source may not be added to this phase. An aqueous solution containing the substrate selected for the production of enzymes is prepared at a concentration of 200-250 g.l⁻¹; this solution must contain the inductive substrate. It is injected after exhaustion of the initial substrate so as to supply an optimized amount ranging between 35 and 45 mg.g⁻¹ cells (fed batch). The residual sugar concentration in the culture medium is below 1 g.l⁻¹ during this fed batch stage.

The enzymatic hydrolysis stage is followed by a fermentation stage, then by a stage of distillation and separation of the alcohol obtained.

In the case of ethanolic fermentation, the alcohol obtained is ethanol.

In the case of an acetonobutylic or ABE type fermentation, a mixture of alcohols consisting of butanol and ethanol is obtained at the end of the process. This type of fermentation is achieved by bacteria belonging to the Clostridium genus, more specifically to the Clostridium acetobutylicum species.

Similarly, if the fermentation is of IBE type using Clostridium bejerinckii strains, a mixture consisting of isopropanol, butanol and ethanol is obtained.

The enzymatic hydrolysis and fermentation stages can be carried out simultaneously (SSF process—Simultaneous Saccharification and Fermentation), and they are followed by a stage of distillation and separation of the alcohol obtained.

Example 1 is given by way of comparison and Example 2 illustrates the invention without limiting the scope thereof.

### Example 1

### Production of Enzymes on Lactose

The production of cellulases was carried out in a mechanically stirred fermenter. The medium had the following composition: KOH 1.66 g.l⁻¹, 85% H₃PO₄ 2 ml.l⁻¹, (NH4)₂SO₄ 2.8 g.l⁻¹, MgSO₄, 7H₂O 0.6 g.l⁻¹, CaCL₂ 0.6 g.l⁻¹, MnSO₄ 3.2 mg.l⁻¹, ZnSO₄, 7H₂O 2.8 mg.l⁻¹, CoCl₂ 4.0 mg.l⁻¹, FeSO₄, 7H₂O 10 mg.l⁻¹, Corn Steep 1.2 g.L⁻¹, anti-foaming agent 0.5 ml.l⁻¹.

The fermenter containing 1.75 l mineral medium and 70 g lactose was sterilized at 120° C., then seeded with 0.25 l of a liquid preculture of the CL847 Trichoderma reesei strain. The preculture medium, supplemented with 5 g.L⁻¹ potassium phthalate to control the pH variations, was identical to that of the fermenter. The preculture fungus was grown on lactose, at a concentration of 30 g.l⁻¹. The inoculum growth lasted for 2 to 3 days and was carried out between 27° C. and 30° C. on a shaker table.

After 46 hours growth, the initial substrate of the fermenter was exhausted and the 250 g.l⁻¹ lactose solution was continuously injected at a flow rate of 4.5 ml.h⁻¹ up to 142 hours.

The temperature was set at 27° C. during the biomass growth stage, then to 25° C. until the end of the cultivation. The pH value was set at 5 during the growth stage, then to 4 until the end of the cultivation by addition of an ammonia solution that provided the nitrogen required for synthesis of the excreted proteins. The dissolved oxygen content was kept above 15 to 20% by adjusting aeration and stirring.

Enzyme production was followed by measuring the extracellular proteins by means of the Folin (Lowry) method, after mycelium separation by filtering or centrifuging. The cellulolytic activities were determined by means of the filter paper activity method (FPU: filter paper unit) for overall activity and cellobiase activity, an activity considered to limit the process of enzymatic hydrolysis of the lignocellulosic biomass. The FPU activity was measured on Whatman No.1 filter paper at an initial concentration of 50 g.l⁻¹; the test sample of the enzymatic solution to be analyzed that released the equivalent of 2 g.l⁻¹ glucose (colorimetric determination) in 60 minutes was determined. The cellobiase activity was measured on cellobiose at a concentration of 20 mM; the test sample releasing 0.5 g.l⁻¹ glucose (enzymatic determination) in 30 minutes was determined.

The activities in U.ml⁻¹ are expressed in micromoles of glucose released per minute and per milliliter of enzymatic solution.

The analytical determinations of the final must gave the following results:

| | |
|---|---|
| Substrate consumed g · l⁻¹ | 79.6 |
| Biomass g · l⁻¹ | 13.5 |
| Proteins mg · ml⁻¹ | 37.8 |
| FPU U · ml⁻¹ | 22.1 |
| Cellobiases U · ml⁻¹ | 25.2 |

### Example 2

### Production of Enzymes on Saccharose

Enzyme production was carried out under the same conditions as in Example 1, but the lactose was replaced, in the batch stage, by saccharose and the fed-batch stage performed with a solution of 60% lactose and 40% saccharose. The strain used was a CL847-derived strain transformed with the A. niger invertase (Berges et al., 1993).

The fermenter containing 1.75 l mineral medium and 40 g pure saccharose was seeded with 0.25 l of a liquid preculture of the CL847 Trichoderma reesei strain. The preculture carbon-containing substrate was glucose at a concentration of 20 g.l⁻¹. After 48 hours growth, after exhaustion of the initial substrate, the 200 g.l⁻¹ solution of 60% lactose and 40% saccharose was continuously injected at a flow rate of 5 ml.h⁻¹ up to 165 hours.

The analytical determinations of the final must gave the following results:

| | |
|---|---|
| Substrate consumed g · l⁻¹ | 71.9 |
| Biomass g · l⁻¹ | 12.2 |
| Proteins mg · ml⁻¹ | 32 |
| FPU U · ml⁻¹ | 23 |
| Cellobiases U · ml⁻¹ | 34 |

The enzyme productions obtained were close in terms of enzymatic activities and yield. These values were compatible with an efficient lignocellulosic biomass enzymatic hydrolysis. This enzymatic hydrolysis and the fermentation gave, for the two examples, similar results in terms of alcohol production.

1) A method of producing alcohol from pretreated lignocellulosic biomass, comprising conducting an enzymatic hydrolysis stage of said lignocellulosic biomass with cellulolytic and/or hemicellulolytic enzymes produced using at least one effluent from another ethanol production process using a sugar plant as the feedstock.

2) A method as claimed in claim 1, wherein said effluent used for the production of cellulolytic and/or hemicellulolytic enzymes comprises sweet juices resulting from beet or sugar cane washing, and/or sugar refinery molasses.

3) A method as claimed in claim 1, wherein the production of cellulolytic and/or hemicellulolytic enzymes is achieved with cellulolytic micro-organism strains, using for its growth saccharose in natural or modified form as the carbon source.

4) A method as claimed in claim 3, wherein the production of cellulolytic and/or hemicellulolytic enzymes is achieved with fungi also using as the carbon source another industrial soluble sugar or a hemicellulosic fraction extract in form of monomers resulting from the pretreated biomass, or a fermentation vinasse extract from lignocellulosic biomass hydrolysis products, or a mixture thereof.

5) A method as claimed in claim 3, wherein the microorganism strains belongs to the Trichoderma, Aspergillus, Penicillium or Schizophyllum genera.

6) A method as claimed in claim 5, wherein the microorganism belongs to the Trichoderma reesei species.

7) A method as claimed in claim 1, wherein the lignocellulosic biomass is selected from among straws, wood, forest crops, alcohol-producing, sugar crop and cereal crop residues, paper industry residues, cellulosic and lignocellulosic material transformation products.

8) A method as claimed in claim 1, wherein the enzymatic hydrolysis stage is followed by a fermentation stage, then by a stage of distillation and separation of the alcohol.

9) A method as claimed in claim 8, wherein the fermentation stage is of ethanolic fermentation type and the alcohol obtained is ethanol.

10) A method as claimed in claim 8, wherein the fermentation stage is of ABE fermentation type and the alcohol obtained is a mixture of butanol and ethanol.

11) A method as claimed in claim 8, wherein the fermentation stage is of IBE fermentation type and the alcohol obtained is a mixture of butanol, ethanol and isopropanol.

12) A method as claimed in claim 1, wherein said enzymatic hydrolysis stage and a fermentation stage are carried out simultaneously and are followed by a stage of distillation and separation of the alcohol.

13) A method according to claim 1, wherein said effluent results from beet sugar washing.

14) A method according to claim 1, wherein said effluent results from cane sugar washing.

15) A method according to claim 1, wherein said effluent results from sugar refinery molasses.

16) A method as claimed in claim 2, wherein the lignocellulosic biomass is selected from among straws, wood, forest crops, alcohol-producing sugar crop and cereal crop residues, paper industry residues, cellulosic and lignocellulosic material transformation products.
